(19) United States
(12) Patent Application Publication
(10) Pub. No.: US 2025/0339487 A1
(43) Pub. Date: Nov. 6, 2025

(54) USE OF CITRUS DEPRESSA EXTRACT FOR PREPARING COMPOSITION USED IN REPAIRING AND/OR RELIEVING CORNEAL NERVE INJURY

(71) Applicant: Chung Shan Medical University, Taichung City (TW)

(72) Inventors: PEI-CHENG LIN, Taichung City (TW); CHIA-WEN HOU, Tainan City (TW); PEI-YU WU, Kaohsiung City (TW); HAN-HSIN CHANG, Taichung City (TW); CHAO-SHENG HU, Taichung City (TW)

(73) Assignee: Chung Shan Medical University, Taichung City (TW)

(21) Appl. No.: 19/197,354

(22) Filed: May 2, 2025

(30) Foreign Application Priority Data
May 3, 2024 (TW) 113116485

(51) Int. Cl. A61K 36/752; A61K 31/714; A61P 27/02

(52) U.S. Cl. CPC A61K 36/752; A61K 31/714; A61P 27/02

(57) ABSTRACT

A use of a Citrus depressa extract in preparing a composition used in repairing and/or relieving corneal nerve injury, wherein the composition includes a Citrus depressa extract in an effective amount. The Citrus depressa extract of the composition is a natural plant ingredient and does not cause side effects in a series of animal experiments. When a subject is orally administered the composition, the Citrus depressa extract is configured to provide effects of relieving and/or repairing cornea injury of the subject, including promoting corneal wound, increasing tear secretion, and promoting and/or improving regeneration of corneal nerves, thereby protecting eyes and repairing cornea injury.

### BACKGROUND OF THE INVENTION

### Technical Field

The present invention relates generally to uses of Citrus depressa, and more particularly to the use of a Citrus depressa extract for preparing a composition used in repairing and/or relieving corneal nerve injury.

### Description of Related Art

In recent years, due to the widespread use of 3C (Computers, Communications, and Consumer Electronics) products, the number of patients suffering from pathological myopia, eye aging, and eye diseases has been increasing. The cornea is a tissue with a high density of nerves. When the cornea of the eye is injured, lesions and damage to the corneal nerves easily occur, wherein lesions and damage to the corneal nerves are caused by viral infection, laser eye surgery for myopia, dry eye syndrome, and other conditions.

Every year, many patients undergo cornea transplant surgery. However, even after undergoing cornea transplantation, some patients suffer from poor healing and repair of the cornea and the cornea nerves due to prior corneal nerve damage and the adverse effects of the transplant surgery.

The conventional clinical way to regenerate and heal the corneal nerves is to administer an eye drop containing nerve growth factors (NGF) to the eye surface of the patient by instillation at a fixed dose and a fixed frequency, thereby effectively facilitating nerve regeneration. However, such treatment using the nerve growth factors (NGF) is costly, limiting the widespread use of such treatment among patients.

### BRIEF SUMMARY OF THE INVENTION

In view of the above, the primary objective of the present invention is to provide a use of a Citrus depressa extract in preparing a composition used in repairing and/or relieving corneal nerve injury, wherein the Citrus depressa extract is a natural plant ingredient and is low in cost. Experiments for the present invention show that the Citrus depressa extract provides the effect of promoting and/or improving the regeneration of corneal nerves in a subject.

The present invention provides a use of a Citrus depressa extract in preparing a composition used in repairing and/or relieving corneal nerve injury, wherein the composition includes the Citrus depressa extract in an effective amount, the composition is orally administered to a subject, and the Citrus depressa extract is configured to promote and/or improve regeneration of corneal nerves of the subject.

In an embodiment, the effective amount of the Citrus depressa extract is between 6 g/kg body weight and 12 g/kg body weight, based on the body weight of the subject.

In an embodiment, when the subject suffering from a cornea injury is administered the composition, the Citrus depressa extract is configured to promote corneal wound repair and to increase tear secretion on the cornea.

In an embodiment, the composition is used in combination with an eye drop, and the eye drop is administered to the eye of the subject through instillation.

In an embodiment, the eye drop contains vitamin B12, with the content of the vitamin B12 accounting for between 0.05 wt % and 0.1 wt % of the total content of the eye drop, and a usage dose of the eye drop is between 5 μl and 10 μl.

In an embodiment, when the subject suffering from a cornea injury is administered the composition and the eye drop, the Citrus depressa extract and the vitamin B12 are configured to promote corneal wound repair and to increase tear secretion on the cornea surface.

With the aforementioned design, the Citrus depressa extract of the composition is a natural plant ingredient and does not cause side effects in a series of animal experiments. When the subject is orally administered the composition, the Citrus depressa extract in the effective amount could provide the effects of relieving and/or repairing cornea injury of the subject, including promoting corneal nerve repair, increasing tear secretion, and promoting and/or improving regeneration of corneal nerves. The use of the composition in combination with the eye drop including the vitamin B12 could also provide the effects of protecting eyes and repairing corneal injury.

Moreover, the Citrus depressa extract is low in cost. The Citrus depressa extract could actually provide the effects of healing corneal injury. The composition of the present invention could provide a potential for replacing conventional chemical eye drops in human experiments in the future.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 is a schematic view showing the experiment model used in examining the effect of the composition, including the Citrus depressa extract for repairing and/or relieving corneal nerve injury according to an embodiment of the present invention;

FIG. 2 is a data chart showing the tear secretions of the normal control group, the vehicle group, and the experimental groups 1 to 3 on different days according to the embodiment of the present invention;

FIG. 3 is a schematic view showing the images of the stained ocular surface epithelium injury of the eyes of the subjects of the normal control group, the vehicle group, and the experimental groups 1 to 3 on different days according to the embodiment of the present invention;

FIG. 4 is a data chart showing the cornea injury areas of the subjects of the normal control group, the vehicle group, and the experimental groups 1 to 3 measured on different days according to the embodiment of the present invention;

FIG. 5 is a schematic view showing the micrographs of the corneal nerves of the subjects of the normal control group, the vehicle group, and the experimental groups 1 to 3 taken on different days according to the embodiment of the present invention;

FIG. 5A is a schematic view showing the micrographs of the corneal nerves of the subjects of the normal control group, the vehicle group, and the experimental groups 1 to 3 taken on Day 0 of the experiment according to the embodiment of the present invention;

FIG. 5B is a schematic view showing the micrographs of the corneal nerves of the subjects of the normal control group, the vehicle group, and the experimental groups 1 to 3 taken on Day 7 of the experiment according to the embodiment of the present invention;

FIG. 5C is a schematic view showing the micrographs of the corneal nerves of the subjects of the normal control group, the vehicle group, and the experimental groups 1 to 3 taken on Day 14 of the experiment according to the embodiment of the present invention;

FIG. 5D is a schematic view showing the micrographs of the corneal nerves of the subjects of the normal control group, the vehicle group, and the experimental groups 1 to 3 taken on Day 21 of the experiment according to the embodiment of the present invention;

FIG. 6 is a data chart showing the corneal nerve densities of the subjects of the normal control group, the vehicle group, and the experimental groups 1 to 3 according to the embodiment of the present invention;

FIG. 7A is a schematic view showing the images of the stained corneal tissues of the subjects of the normal control group, the vehicle group, and the experimental groups 1 to 3 taken on different days according to the embodiment of the present invention;

FIG. 7B is a bar chart showing the areas of the stained corneal tissues of the subjects of the normal control group, the vehicle group, and the experimental groups 1 to 3 measured on different days according to the embodiment of the present invention;

FIG. 8A is a schematic view showing the images of the stained antibody-binding tissues of the subjects of the normal control group, the vehicle group, and the experimental groups 1 to 3 taken on different days according to the embodiment of the present invention; and

FIG. 8B is a data chart showing the areas of the stained antibody-binding tissues of the subjects of the normal control group, the vehicle group, and the experimental groups 1 to 3 measured on different days according to the embodiment of the present invention.

### DETAILED DESCRIPTION OF THE INVENTION

The present invention provides an embodiment of the use of a Citrus depressa extract in preparing a composition used in repairing and/or relieving corneal nerve injury. The composition includes the Citrus depressa extract in an effective amount. In the current embodiment, the Citrus depressa extract is obtained by extracting the peels of Citrus depressa. The peels of Citrus depressa are ground and triturated. The peels are mixed with alcohol and are oscillated at room temperature. After oscillation, two filtrations are performed by using a filtering bag and a filter paper. A filtrate obtained through the filtrations is concentrated under reduced pressure to yield the Citrus depressa extract. The Citrus depressa extract includes ingredients such as nobiletin, tangeretin, sinensetin, and hesperidin. The Citrus depressa extract has functions such as anti-inflammation and anti-oxidation.

In the current embodiment, the composition is orally administered to a subject three times a day for 3 to 14 consecutive days. The effective amount of the Citrus depressa extract is between 6 g/kg body weight and 12 g/kg body weight based on the body weight of the subject (mouse model). The effective amount of the Citrus depressa extract could be adjusted based on the effect and the purpose of the experiment. In an embodiment, the effective amount of the Citrus depressa extract is between 6.88 g/kg body weight and 11.44 g/kg body weight based on the body weight of the subject (mouse model). In the current experiment, when the subject suffering from cornea injury is administered the composition, the Citrus depressa extract could significantly promote and/or improve regeneration of corneal nerves of the subject, and promote corneal wound repair and increase tear secretion.

In another embodiment, the composition could be administered in combination with an eye drop. The eye drop is administered to the subject through instillation administration, wherein the eye drop is instilled on the eye surface of the subject after the subject is orally administered the composition. The eye drop is administered three times a day for 3 to 14 consecutive days in accordance with the administration days of the composition. The eye drop contains vitamin B12. The content of vitamin B12 accounts for between 0.05 wt % and 0.1 wt % of the total content of the eye drop. A usage dose of the eye drop is between 5 μl and 10 μl per instillation. The current experiment discovers that when a subject suffering from corneal injury is administered the composition and the eye drop, the Citrus depressa extract and the vitamin B12 cooperate to promote corneal wound repair and increase tear secretion at the cornea.

The term “effective amount” as referred to herein is an amount of an active ingredient in a composition which is sufficient to produce a desired physiological response. The effective amount of the active ingredient does not necessarily cure a disease or a symptom but could slow, stop, prevent, or relieve the development of the symptom. The actual effective amount of the active ingredient depends on many factors, such as the particular condition of the experiment, the physiological condition of subjects (e.g., weight, age, gender), the type of subjects undertaking experiments, the duration of experiments, and the actual formula used. For example, the effective amount of the active ingredient could be expressed in grams, milligrams, or micrograms. Alternatively, the effective amount of the active ingredient could be expressed in a ratio of dose to body weight, such as grams per kilogram of body weight (g/kg). Alternatively, the effective amount of the active ingredient could be expressed in concentration, such as molar concentration, weight concentration, volume concentration, mole fraction, mass fraction, and mixing ratio. One of ordinary skill in the art could calculate the human equivalent dose (HED) for human medication based on doses determined from animal models. Alternatively, one of ordinary skill in the art could convert the test dose in an animal model into an equivalent dose (ED) of the composition that could be administered to humans, pets, or mammals.

The term “subject” as referred herein refers to animals such as humans that could receive the composition of the present invention. Unless otherwise specified, the term “subject” refers to a mammal, bird, or fowl that could produce therapeutic benefits after receiving the composition of the present invention, such as human, rat, mouse, guinea pig, monkey, pig, goat, cow, horse, dog, cat, bird, and fowl. In the current embodiment, the term “subject” refers to mice for experiments. The effective amount of the Citrus depressa extract in the experiment of the subject (mouse) as exemplified in the current embodiment could be converted to human equivalent dose (HED) or effective dose for related pets, birds, or fowls.

The content and formula used in the preparing process in the current embodiment is not a limitation of the present invention. The parameters that could be appropriately adjusted by one of ordinary skill in the art after referring to the present invention should still fall within the scope of the present invention.

In order to demonstrate the purpose, the features, and the effects of the present invention, the composition in the current embodiment is administered to the subject, and the cornea injury area, the tear secretion, and the corneal nerve density of the subject are analyzed, thereby illustrating that the Citrus depressa extract could actually provide the effect of repairing and/or relieving cornea injury.

### Establishing an Animal Experiment Model

The “subjects” used in the current experiment are ICR mice purchased from BioLASCO Taiwan Co., Ltd.

The current experiment includes a normal control group, a vehicle group, and experimental groups 1 to 3.

The normal control group: the subjects (mice) are normal and no cornea injury is induced, and the subjects are fed with saline every day.

The vehicle group: the eyes of the subjects are instilled with 0.2% benzalkonium chloride (BAC) one time a day for 7 consecutive days to induce cornea injury; afterwards, the subjects with the induced cornea injury are fed with saline every day for 14 consecutive days.

The experimental group 1: the eyes of the subjects are instilled with 0.2% benzalkonium chloride (BAC) one time a day for 7 consecutive days to induce cornea injury; afterwards, the subjects with the induced cornea injury are fed with the composition at a fixed dose and a fixed frequency for 14 consecutive days; in the current experiment, the effective amount of the Citrus depressa extract of the composition is 6.88 g/kg based on the body weights of the subjects, and the composition is orally administered to the subjects with the induced cornea injury three times a day.

The experimental group 2: the eyes of the subjects are instilled with 0.2% benzalkonium chloride (BAC) one time a day for 7 consecutive days to induce cornea injury; afterwards, the subjects with the induced cornea injury are fed with the composition at a fixed dose and a fixed frequency and are administered the eye drop to the eyes of the subjects through instillation administration for 14 consecutive days; in the current experiment, the effective amount of the Citrus depressa extract of the composition is 6.88 g/kg based on the body weights of the subjects; the composition is orally administered to the subjects with the induced cornea injury three times a day; the content of the vitamin B12 of the eye drop is 0.05 wt %; the usage dose of the eye drop is about 10 μl per time, and the eye drop is instilled on the eye surfaces of the subjects three times a day.

The experimental group 3: the eyes of the subjects are instilled with 0.2% benzalkonium chloride (BAC) one time a day for 7 consecutive days to induce cornea injury; afterwards, the eyes of the subjects with the induced cornea injury are administered a solution having nerve growth factors at a fixed dose and a fixed frequency for 14 consecutive days; in the current experiment, a usage dose of the solution having the nerve growth factors is 5 μl per time, and the solution having the nerve growth factors is instilled on the eye surfaces of the subjects three times a day.

### Establishing an Experimental Model

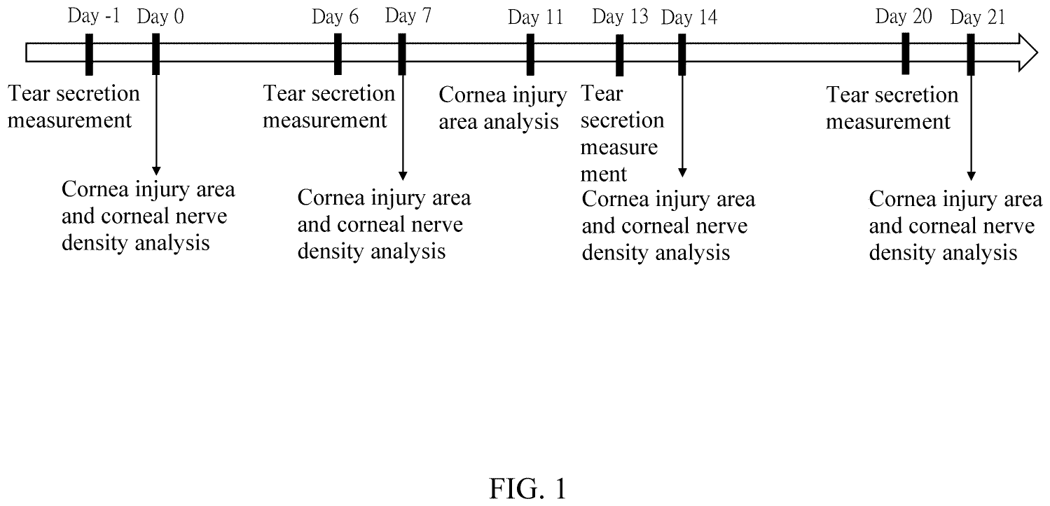

Referring to FIG. 1, Day 0 is the day when the subjects of the normal control group, the vehicle group, and the experimental groups 1 to 3 start undertaking a cornea injury induction; Day −1 is 1 day before the subjects of the vehicle group and the experimental groups 1 to 3 start undertaking the cornea injury induction (Day 0); Day 6 is the sixth day after the subjects of the vehicle group and the experimental groups 1 to 3 start undertaking the cornea injury induction (Day 0); Day 7 is the seventh day after the subjects of the vehicle group and the experimental groups 1 to 3 start undertaking the cornea injury induction (Day 0), and the subjects stop undertaking the cornea injury induction on Day 7; the subjects of the experimental groups 1 to 3 start undertaking a treatment test, wherein the subjects of the experimental group 1 start being administered the composition, the subjects of the experimental group 2 start being administered the composition and the eye drop, the subjects of the experimental group 3 start being administered the solution having the nerve growth factors, and the subjects of the vehicle group start being administered saline.

Day 11 is the eleventh day after the subjects of the vehicle group and the experimental groups 1 to 3 start undertaking the cornea injury induction (Day 0), i.e., the fourth day after the subjects of the experimental groups 1 to 3 start undertaking the treatment test (Day 7); Day 13 is the thirteenth day after the subjects of the vehicle group and the experimental groups 1 to 3 start undertaking the cornea injury induction (Day 0), i.e., the sixth day after the subjects of the experimental groups 1 to 3 start the treatment test (Day 7); Day 14 is the fourteenth day after the subjects of the vehicle group and the experimental groups 1 to 3 start undertaking the cornea injury induction (Day 0), i.e., the seventh day after the subjects of the experimental groups 1 to 3 start undertaking the treatment test (Day 7); Day 20 is the twentieth day after the subjects of the vehicle group and the experimental groups 1 to 3 start undertaking the cornea injury induction (Day 0), i.e., the thirteenth day after the subjects of the experimental groups 1 to 3 start undertaking the treatment test (Day 13); Day 21 is the twenty-first day after the subjects of the vehicle group and the experimental groups 1 to 3 start undertaking the cornea injury induction (Day 0), and the subjects of the experimental groups 1 to 3 stop undertaking the treatment test.

In the current experiment, the subjects of the normal control group, the vehicle group, and the experimental groups 1 to 3 undertake a tear secretion measurement on Day −1, Day 6, Day 13, and Day 20 respectively; the subjects of the normal control group, the vehicle group, and the experimental groups 1 to 3 undertake an ocular surface epithelium injury staining test on Day 0, Day 7, Day 11, Day 14, and Day 21 respectively; the subjects of the normal control group, the vehicle group, and the experimental groups 1 to 3 undertake a corneal nerve staining test on Day 0, Day 7, Day 14, and Day 21 respectively.

### The Tear Secretion Measurement

The subjects of the normal control group, the vehicle group, and the experimental groups 1 to 3 are anesthetized and then the lower eyelid are everted; a tear test strip is gently placed at the lower eyelid of the subjects for 20 seconds; afterwards, the tear test strip is withdrawn from the lower eyelid of the subjects and a pH value and a length of the tear test strip moistened by absorbing tears are measured; the measurement is repeatedly performed on two eyes of the subjects for three times.

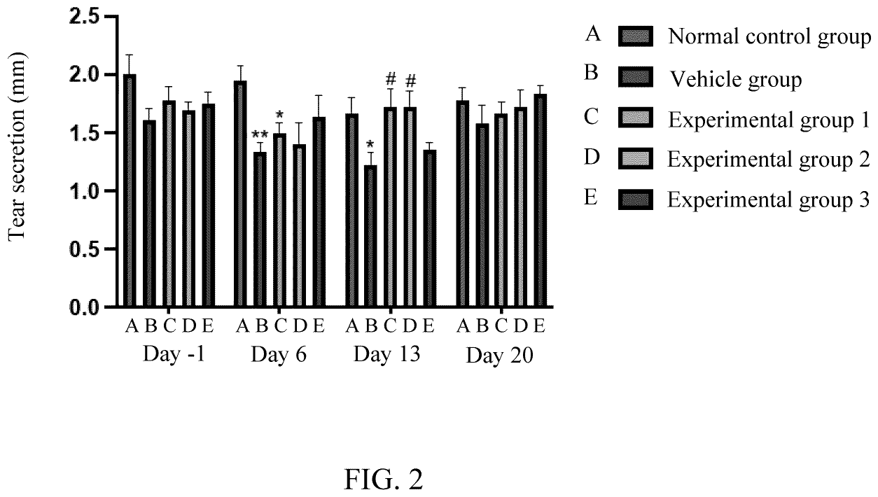

FIG. 2 shows the data chart and the results of the standard error (SE), wherein the results are derived from the measurement values obtained from three independent tests conducted for the subjects (n=3). The symbol “*” refers to a value comparison between the normal control group and the vehicle group or a value comparison between the normal control group and each of the experimental groups 1 to 3. The symbol “#” refers to a value comparison between each of the experimental groups 1 to 3 and the vehicle group, and p<0.05.

Referring to the results in FIG. 2, the subjects of the normal control group, the vehicle group, and the experimental groups 1 to 3 undertake the tear secretion measurement on Day −1, Day 6, Day 13, and Day 20, respectively. On Day −1, the tear secretion of the subjects of the normal control group, the tear secretion of the subjects of the vehicle group, and the tear secretion of the subjects of the experimental groups 1 to 3 measured are not significantly different from one another. On Day 6, the tear secretion of the subjects of the vehicle group and the tear secretion of the subjects of the experimental groups 1 to 3 measured are significantly lower than the tear secretion of the subjects of the normal control group measured, indicating that after the subjects of the vehicle group and the experimental groups 1 to 3 undertake the cornea injury, the tear secretion at the cornea is significantly reduced.

On Day 13, the tear secretion of the subjects of each of the experimental groups 1 to 3 measured increases compared to the tear secretion of the subjects of the vehicle group, wherein the tear secretions of the subjects of the experimental group 1 and the experimental group 2 increase significantly, indicating that the subjects of the experimental group 1 could experience a significant increase of the tear secretion after being administered the composition, and the subjects of the experimental group 2 could experience a significant increase of the tear secretion after being administered the composition and the eye drop. On the sixth day after the experimental groups 1 to 3 start undertaking the treatment test, the experimental groups 1 and 2 show a more pronounced effect of promoting the tear secretion compared to the experimental group 3.

On Day 20, the tear secretions of the subjects of the vehicle group and the experimental groups 1 to 3 measured significantly increase, and the tear secretion of the subjects of the normal control group measured is not significantly different from the tear secretions of the subjects of the vehicle group and the experimental groups 1 to 3, indicating that the cornea of the subjects of the vehicle group are healed and the tear secretion of the subjects of the vehicle group increases accordingly, and the subjects of the experimental groups 1 to 3 could experience the significant increase of the tear secretion after the treatment test.

### Determining the Corneal Injury of the Eyes of the Subjects (Mice)

The subjects of the normal control group, the vehicle group, and the experimental groups 1 to 3 are anesthetized and then a cornea injury staining test is performed; 3.5 ul of lissamine green stain is instilled on the eye surfaces of the subjects, after 30 seconds, blinking is manually performed on the subjects, so that the lissamine green stain is evenly distributed on the ocular surface; finally, the subjects undertaking the cornea injury staining test are respectively placed under a dissection microscope for photographing the ocular surface, so that a health condition of a cornea surface of the subjects could be evaluated; when injuries, defects, or lesions occur on the cornea surface of the subjects, the lissamine green stain forms a stained region on the cornea surface of the subjects, thereby determining changes of the severities of the cornea injury of the subjects.

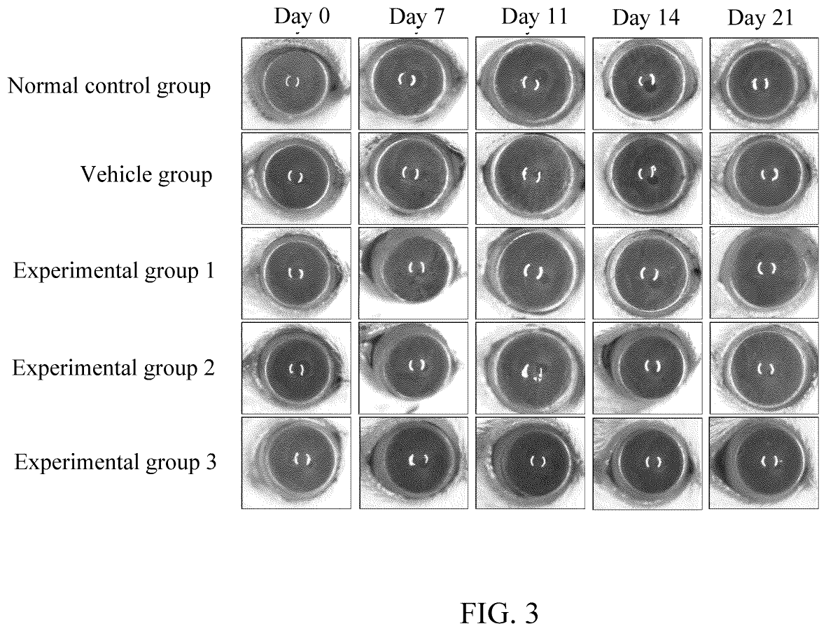

FIG. 3 shows the images of the corneas of the subjects of the normal control group, the vehicle group, and the experimental groups 1 to 3. The images in the first column in FIG. 3 from top to bottom are sequentially the images of the corneas of the subjects of the normal control group, the vehicle group, and the experimental groups 1 to 3 taken on Day 0; it could be seen from the images taken on Day 0 that the ocular surfaces of the subjects of the normal control group, the vehicle group, and the experimental groups 1 to 3 do not have a stained region. The images in the second column in FIG. 3 from top to bottom are sequentially the images of the corneas of the normal control group, the vehicle group, and the experimental groups 1 to 3 taken on Day 7; it could be seen from the images taken on Day 7 that the ocular surfaces of the subjects of the vehicle group and the experimental groups 1 to 3 have clear stained regions, indicating that the cornea injuries of the subjects of the vehicle group and the experimental groups 1 to 3 are actually induced. The images in the third column in FIG. 3 from top to bottom are sequentially the images of the corneas of the subjects of the normal control group, the vehicle group, and the experimental groups 1 to 3 taken on Day 11; it could be seen from the images taken on Day 11 that although the ocular surfaces of the subjects of the vehicle group and the experimental groups 1 to 3 have the stained regions, the stained regions on the ocular surfaces of the subjects of the experimental groups 1 to 3 are smaller than the stained regions of the ocular surfaces of the subjects of the vehicle group, indicating that the severities of the cornea injuries of the subjects of the experimental groups 1 to 3 is significantly reduced.

The images in the fourth column in FIG. 3 from top to bottom are sequentially the images of the corneas of the subjects of the normal control group, the vehicle group, and the experimental groups 1 to 3 taken on Day 14; it could be seen from the images taken on Day 14 that the ocular surfaces of the subjects of the vehicle group and the experimental group 1 to 3 do not have the stained regions, indicating that the severities of the cornea injuries of the subjects of the vehicle group and the experimental groups 1 to 3 are significantly reduced. The images in the fifth column in FIG. 3 from top to bottom are sequentially the images of the corneas of the subjects of the normal control group, the vehicle group, and the experimental groups 1 to 3 taken on Day 21; it could be seen form the images taken on Day 21 that the ocular surfaces of the subjects of the vehicle group and the experimental groups 1 to 3 do not have the stained regions, indicating that the corneas of the subjects of the vehicle group and the experimental groups 1 to 3 are recovered.

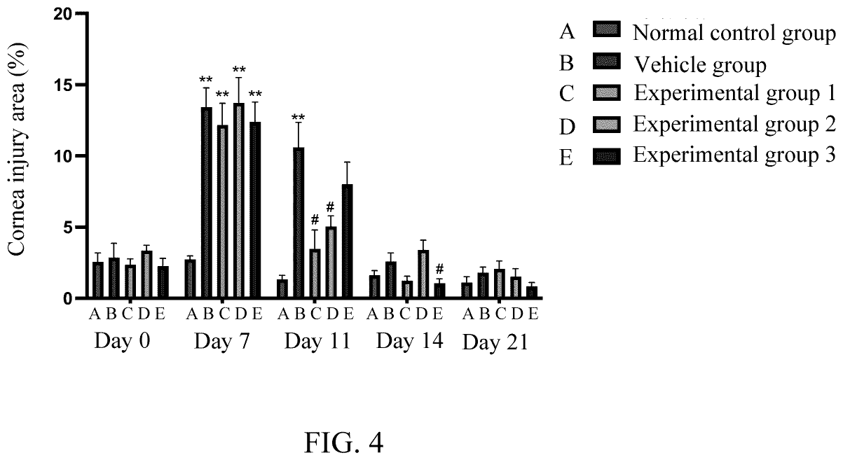

FIG. 4 is a data chart showing the cornea injury areas of the subjects of the normal control group, the vehicle group, and the experimental groups 1 to 3 in FIG. 3 were measured on different days. Percentages of areas of the ocular surfaces being occupied by the stained regions in the eyes of the subjects of the normal control group, the vehicle group, and the experimental groups 1-3 in FIG. 3 are calculated by using Image J.

FIG. 4 shows the data chart and the results of the standard error (SE), wherein the results are derived from the measurement values obtained from three independent tests conducted for the subjects (n=3). The symbol “*” refers to a value comparison between the normal control group and the vehicle group or a value comparison between the normal control group and each of the experimental groups 1 to 3. The symbol “#” refers to a value comparison between each of the experimental groups 1 to 3 and the vehicle group, and p<0.05.

FIG. 4 shows that on Day 0, values of the cornea injury areas of the subjects of the normal control group, the vehicle group, and the experimental groups 1 to 3 are not significantly different from one another. FIG. 4 shows that on Day 7, the values of the cornea injury areas of the subjects of the vehicle group and the experimental groups 1 to 3 increase significantly compared to the value of the cornea injury area of the subjects of the normal control group, indicating that the cornea injury areas of the subjects of the vehicle group and the experimental groups 1 to 3 increase. FIG. 4 shows that on Day 11, the values of the cornea injury areas of the subjects of the experimental groups 1 to 3 significantly decrease compared to the value of the cornea injury area of the subjects of the vehicle group, indicating that the cornea injuries of the subjects of the experimental groups 1 to 3 gradually heal; the values of the cornea injury areas of the subjects of the experimental groups 1 and 2 decrease more significantly; accordingly, the subjects of the experimental group 1 could experience a pronounced healing of the cornea injury after being administered the composition, and the subjects of the experimental group 2 could experience a pronounced healing of the cornea injury after being administered the composition and the eye drop; on the fourth day after the experimental groups 1, 2, and 3 start undertaking the treatment test, the experimental groups 1 and 2 could have a more pronounced effect of relieving the cornea injury compared to the experimental group 3. FIG. 4 shows that on Day 14 and Day 21, the values of the cornea injury areas of the subjects of the vehicle group and the experimental groups 1 to 3 decrease more significantly, indicating that the severities of the cornea injuries of the subjects of the vehicle group and the experimental groups 1 to 3 are significantly reduced.

In summary, the subjects of the experimental groups 1 and 2 administered the composition could experience a rapid healing of the cornea injury on the fourth day after undertaking the treatment test, and the healings of the cornea injuries of the experimental groups 1 and 2 are more efficient than the healing of the cornea injury of the experimental group 3, wherein the decrease of the cornea injury area of the subject of the experimental group 1 is the most significant. Accordingly, the Citrus depressa extract could rapidly promote the healing of the cornea within 5 days after administration.

### Determining the Regeneration of the Corneal Nerves of the Subjects (Mice)

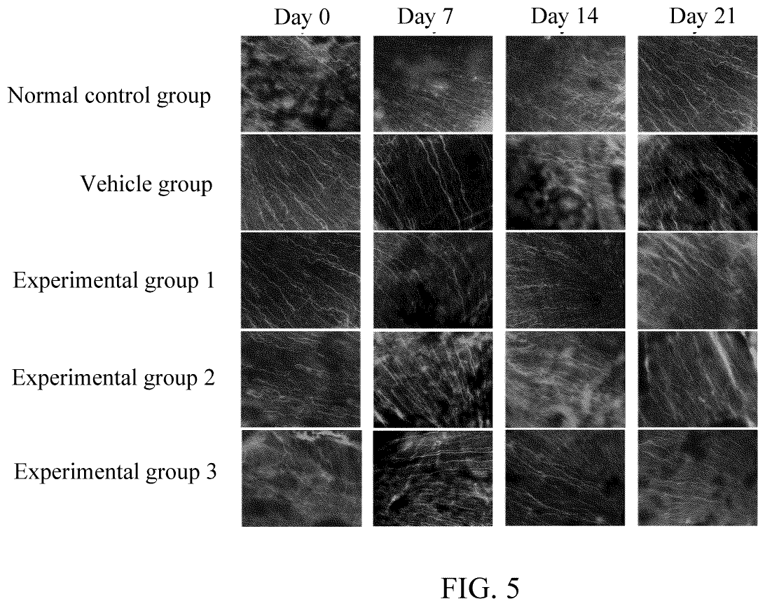

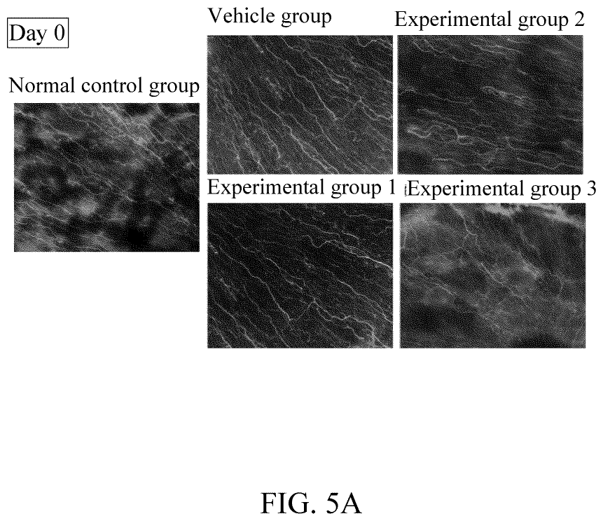

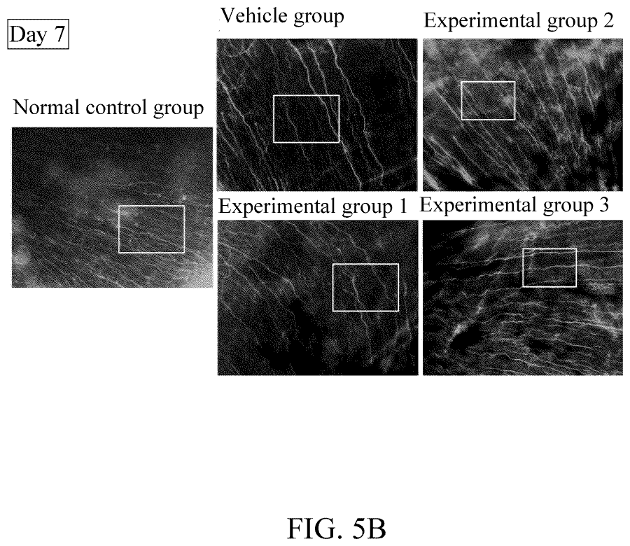

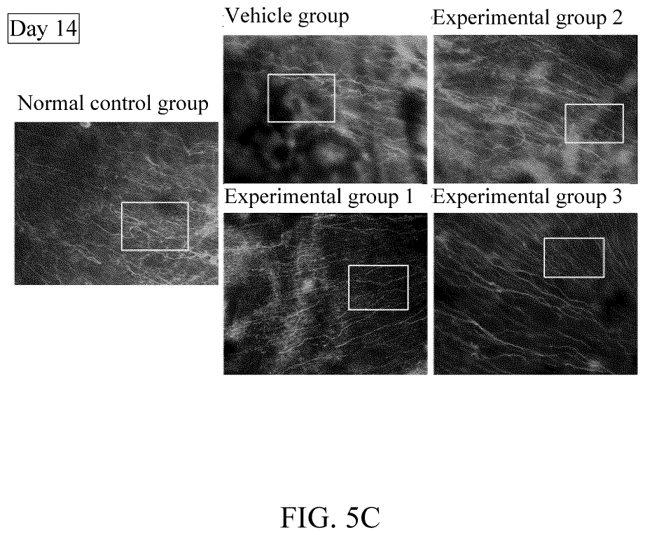

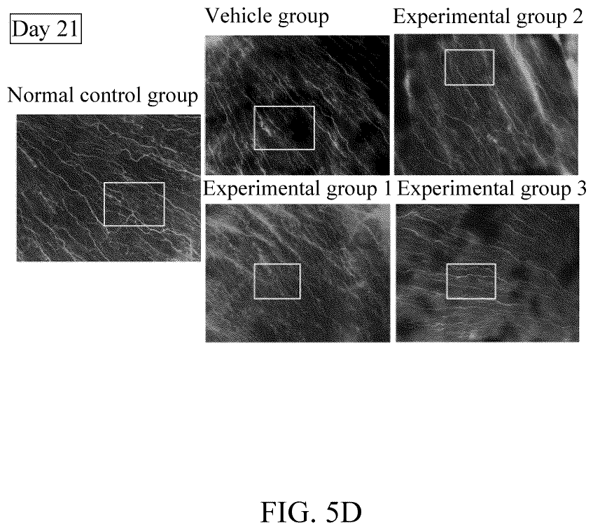

The subjects of the normal control group, the vehicle group, and the experimental groups 1 to 3 are sacrificed and the corneas are removed for staining. A stain used in staining is BIII Tubulin. After staining, the subjects are placed under an upright fluorescence microscope for photographing to obtain the images of the corneal nerves of the normal control group, the vehicle group, and the experimental groups 1 to 3 (referring to FIG. 5). Referring to FIG. 5A to FIG. 5D, under the same experimental day, frame lines are automatically formed on the images of the corneal nerves of the normal control group, the vehicle group, and the experimental groups 1 to 3 through a computer, thereby indicating that the images of the corneal nerves of the normal control group, the vehicle group, and the experimental groups 1 to 3 have regions that are different from others.

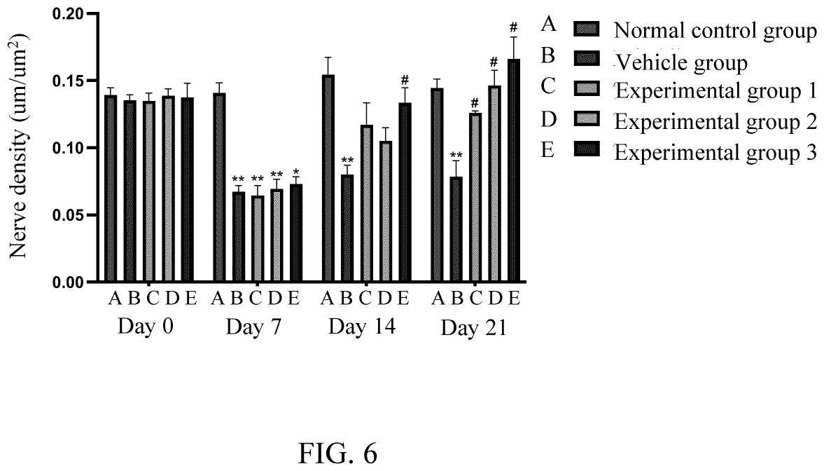

Moreover, FIG. 6 is a data chart showing the corneal nerve densities of the subjects of the normal control group, the vehicle group, and the experimental groups 1 to 3 measured on different days shown in FIG. 5. Total nerve lengths within the same area in the images of the corneal nerves of the normal control group, the vehicle group, and the experimental groups 1 to 3 in FIG. 5 are calculated by using Image J.

FIG. 6 shows the data chart and the results of the standard error (SE), wherein the results are derived from the measurement values obtained from three independent experiments (n=3). The symbol “*” refers to a value comparison between the normal control group and the vehicle group or a value comparison between the normal control group and each of the experimental groups 1 to 3. The symbol “#” refers to a value comparison between each of the experimental groups 1 to 3 and the vehicle group, and p<0.05.

Referring to FIG. 5A and FIG. 6, on Day 0, values of the corneal nerve densities of the normal control group, the vehicle group, and the experimental groups 1 to 3 are not significantly different from one another. Referring to FIG. 5B and FIG. 6, on Day 7, the values of the corneal nerve densities of the vehicle group and the experimental groups 1 to 3 decrease compared to the value of the corneal nerve density of the normal control group, indicating that the subjects of the vehicle group and the experimental groups 1 to 3 suffer from the corneal nerve injury due to undertaking the cornea injury induction. Referring to FIG. 5C and FIG. 6, on Day 14, the values of the corneal nerve densities of the experimental groups 1 to 3 increase compared to the value of the corneal nerve density of the vehicle group, indicating that the corneal nerves of the subjects of the experimental groups 1 to 3 start performing hyperplasia. Referring to FIG. 5D and FIG. 6, on Day 21, the values of the corneal nerve densities of the experimental groups 1 to 3 significantly increase compared to the value of the corneal nerve density of the vehicle group, and the values of the corneal nerve densities of the experimental groups 1 to 3 are similar to the value of the corneal nerve density of the normal control group, indicating that the corneal nerves of the subjects of the experimental groups 1 to 3 heal. Accordingly, the subjects of the experimental groups 1 and 2 could experience a promoted corneal nerve repair after being administered the composition.

### Determining the Fibrosis of the Corneal Tissues of the Subjects (Mice)

The subjects of the normal control group, the vehicle group, and the experimental groups 1 to 3 are sacrificed and undergo corneal sectioning. The sectioned corneal tissues are stained using H&E stain. The staining antibody is α-smooth muscle actin (α-SMA). Finally, after staining, the sectioned corneal tissues of the subjects are placed under a dissection microscope for photographing and recording to evaluate changes in the fibrosis of the corneal tissues of the subjects caused by the injuries to the corneal tissues.

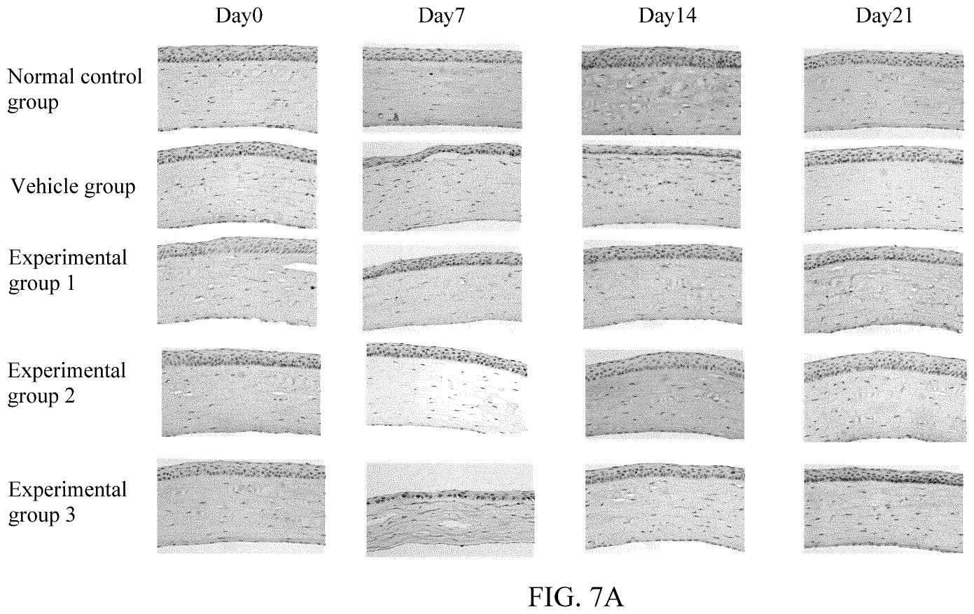

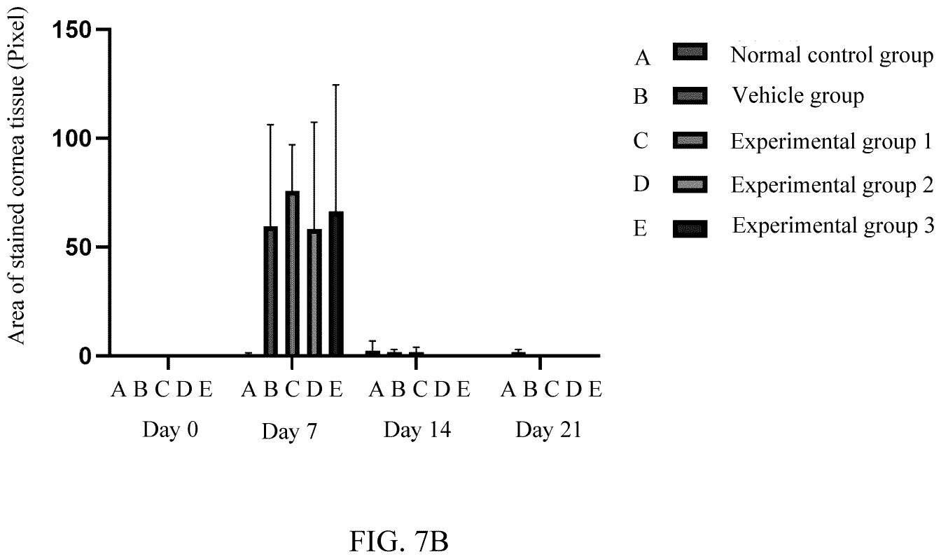

FIG. 7A shows the images of the corneal tissues of the subjects of the normal control group, the vehicle group, and the experimental groups 1 to 3. FIG. 7A should be seen along with the data chart in FIG. 7B showing the areas of the stained corneal tissues. The images in the first column in FIG. 7A from top to bottom are sequentially the images of the corneal tissues of the subjects of the normal control group, the vehicle group, and the experimental groups 1 to 3 taken on Day 0. Referring to FIG. 7A and FIG. 7B, the corneal tissues of the subjects of the normal control group, the vehicle group, and the experimental groups 1 to 3 do not have stained regions on Day 0. The images in the second column in FIG. 7A from top to bottom are sequentially the images of the corneal tissues of the subjects of the normal control group, the vehicle group, and the experimental groups 1 to 3 taken on Day 7. Referring to FIG. 7A and FIG. 7B, the corneal tissues of the subjects of the vehicle group and the experimental groups 1 to 3 have the stained regions on Day 7, indicating that the fibrosis is induced in the corneal tissues of the subjects of the vehicle group and the experimental groups 1 to 3; the area of the stained regions of the vehicle group measured is not significantly different from the areas of the stained regions of the experimental groups 1 to 3, indicating that on Day 7, the experimental groups 1 and 2, which are administered the composition, do not experience a relief of the fibrosis of the corneal tissues compared to the vehicle group.

The images in the third column in FIG. 7A from top to bottom are sequentially the images of the corneal tissues of the subjects of the normal control group, the vehicle group, and the experimental groups 1 to 3 taken on Day 14. Referring to FIG. 7A and FIG. 7B, the corneal tissues of the subjects of the vehicle group and the experimental groups 1 to 3 show a decrease in the stained regions, indicating that severities of the fibrosis of the corneal tissues of the subjects of the vehicle group and the experimental groups 1 to 3 are significantly reduced. The images in the fourth column in FIG. 7A from top to bottom are sequentially the images of the corneal tissues of the subjects of the normal control group, the vehicle group, and the experimental groups 1 to 3 taken on Day 21. Referring to FIG. 7A and FIG. 7B, the corneal tissues of the subjects of the vehicle group and the experimental groups 1 to 3 show a greater decrease of the stained regions, indicating that after Day 14, the corneal tissues of the subjects of the vehicle group and the experimental groups 1 to 3 could perform self-healing, thereby relieving the fibrosis of the corneal tissues.

### Determining Prognostic Indicators of the Inflammatory Response and the Fibrosis of the Corneas of the Subjects (Mice)

The subjects of the normal control group, the vehicle group, and the experimental groups 1 to 3 are sacrificed and undergo corneal sectioning. The sectioned corneal tissues are subjected to immunohistochemical staining. The staining antibody is transforming growth factor β1 (TGF-β1). Finally, after staining, the sectioned corneal tissues of the subjects are placed under a dissection microscope for photographing and recording to evaluate changes in the inflammatory response of the subjects caused by the injuries to the antibody-binding tissues

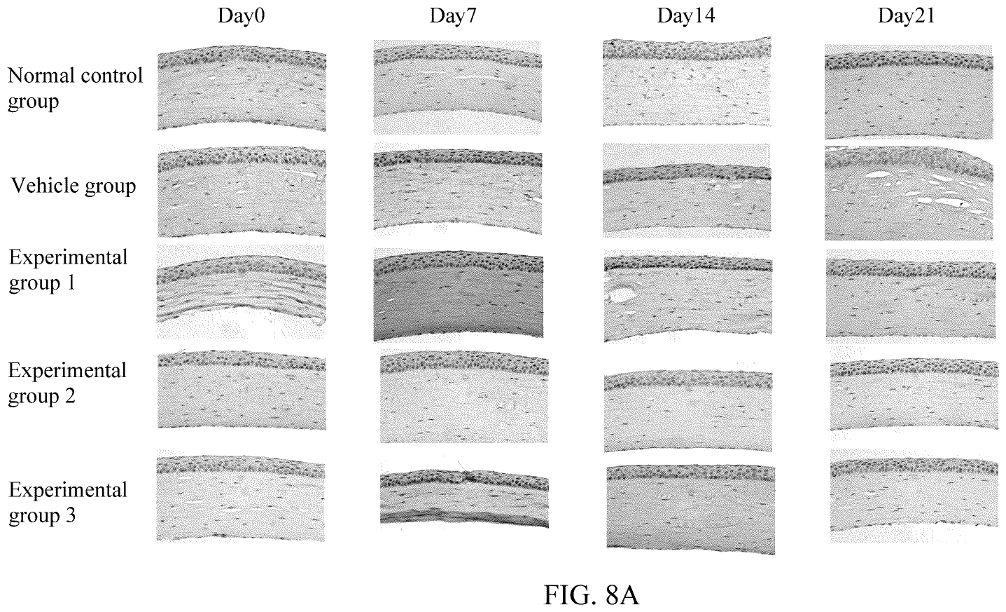

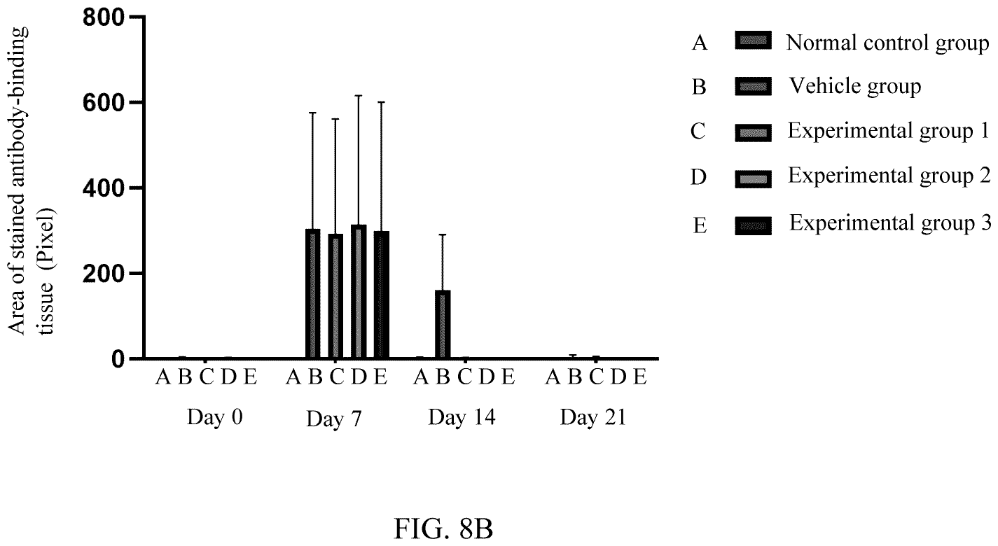

FIG. 8A shows the images of the antibody-binding tissues of the subjects of the normal control group, the vehicle group, and the experimental groups 1 to 3. FIG. 8A should be seen along with the data chart in FIG. 8B showing the areas of the stained regions. The images in the first column in FIG. 8A from top to bottom are sequentially the images of the antibody-binding tissues of the subjects of the normal control group, the vehicle group, and the experimental groups 1 to 3 taken on Day 0. Referring to FIG. 8A and FIG. 8B, the antibody-binding tissues of the subjects of the normal control group, the vehicle group, and the experimental groups 1 to 3 do not have the stained regions on Day 0. The images in the second column in FIG. 8A are the images of the antibody-binding tissues of the subjects of the normal control group, the vehicle group, and the experimental groups 1 to 3 taken on Day 7. Referring to FIG. 8A and FIG. 8B, the antibody-binding tissues of the subjects of the vehicle group and the experimental groups 1 to 3 have the stained regions on Day 7, indicating that the inflammatory response is induced in the antibody-binding tissues of the subjects of the vehicle group and the experimental groups 1 to 3; the area of the stained regions of the vehicle group measured is not significantly different from the areas of the stained regions of the experimental groups 1 to 3; in other words, the experimental groups 1 and 2, which are administered the composition, do not experience a relief of the inflammatory response compared to the vehicle group on Day 7.

The images in the third column in FIG. 8A from top to bottom are sequentially the images of the antibody-binding tissues of the subjects of the normal control group, the vehicle group, and the experimental groups 1 to 3 taken on Day 14. Referring to FIG. 8A and FIG. 8B, the antibody-binding tissues of the subjects of the experimental groups 1 to 3 show a significant decrease in the stained regions compared to the vehicle group, indicating that on Day 14, the subjects of the experimental groups 1 and 2, which are administered the composition, experience a relief of the inflammatory response of the antibody-binding tissues after injuries. The images in the fourth column in FIG. 8A from top to bottom are sequentially the images of the antibody-binding tissues of the subjects of the normal control group, the vehicle group, and the experimental groups 1 to 3 taken on Day 21. Referring to FIG. 8A and FIG. 8B, the antibody-binding tissues of the subjects of the vehicle group and the experimental groups 1 to 3 show a significant decrease in the stained regions, indicating that the antibody-binding tissues of the subjects of the vehicle group could perform self-healing to correspond to the inflammatory response on Day 21.

Accordingly, the Citrus depressa extract of the composition is a natural plant ingredient and does not cause side effects in a series of animal experiments. When the subject is orally administered the composition, the Citrus depressa extract in the effective amount could provide the effects of relieving and/or repairing the cornea injury of the subject, including promoting wound repair of the cornea, increasing tear secretion at the cornea, promoting and/or improving regeneration of the corneal nerves, and healing the inflammatory response of the tissues. The use of the composition in combination with the eye drop containing vitamin B12 could also provide the effects of protecting eyes and repairing cornea injury.

Moreover, the Citrus depressa extract is low in cost. The Citrus depressa extract could actually provide the effects of healing cornea injury. The composition of the present invention could provide the potential to replace conventional chemical eye drops in human experiments in the future.

It must be pointed out that the embodiments described above are only some preferred embodiments of the present invention. All equivalent structures and methods which employ the concepts disclosed in this specification, and the appended claims should fall within the scope of the present invention.

What is claimed is:

1. A use of a Citrus depressa extract in preparing a composition used in repairing and/or relieving corneal nerve injury, wherein the composition comprises the Citrus depressa extract in an effective amount, the composition is orally administered to a subject, and the Citrus depressa extract is configured to promote and/or improve regeneration of corneal nerves of the subject.

2. The use of the Citrus depressa extract in preparing the composition used in repairing and/or relieving corneal nerve injury as claimed in claim 1, wherein the effective amount of the Citrus depressa extract is between 6 g/kg and 12 g/kg based on weight of the subject.

3. The use of the Citrus depressa extract in preparing the composition used in repairing and/or relieving corneal nerve injury as claimed in claim 2, wherein when the subject suffering from a cornea injury is administered the composition, the Citrus depressa extract is configured to promote corneal wound repair and to increase tear secretion.

4. The use of the Citrus depressa extract in preparing the composition used in repairing and/or relieving corneal nerve injury as claimed in claim 2, wherein the composition is used in combination with an eye drop, and the eye drop is administered to an eye of the subject through instillation.

5. The use of the Citrus depressa extract in preparing the composition used in repairing and/or relieving corneal nerve injury as claimed in claim 4, wherein the eye drop contains vitamin B12, a content of the vitamin B12 accounts for between 0.05 wt % and 0.1 wt % of a total content of the eye drop, and a usage dose of the eye drop is between 5 μl and 10 μl.

6. The use of the Citrus depressa extract in preparing the composition used in repairing and/or relieving corneal nerve injury as claimed in claim 5, wherein when the subject suffering from a cornea injury is administered the composition and the eye drop, the Citrus depressa extract and the vitamin B12 are configured to promote corneal wound repair and to increase tear secretion at a cornea.
